(19) United States
(12) Patent Application Publication
(10) Pub. No.: US 2003/0157165 A1
(43) Pub. Date: Aug. 21, 2003

(54) Stable saccharide-free tablets comprising a salt of quinapril or moexipril

(76) Inventor: Bernard Charles Sherman, Willowdale (CA)

Correspondence Address: NIXON & VANDERHYE P.C., 8th Floor, 1100 North Glebe Road, Arlington, VA, 22201-4714, US

(21) Appl. No.: 10/060,191

(22) Filed: Feb. 1, 2002

(51) Int. Cl. A61K 31/47; A61K 9/20

(52) U.S. Cl. 424/465; 514/310

(57) ABSTRACT

Saccharide-free tablets comprising a salt of quinapril or moexipril and an alkaline compound and further comprising an excipient that stabilizes against hydrolysis and is either water-soluble or absorbs water.

### BACKGROUND OF THE INVENTION

Quinapril and Moexipril are known ACE (Angiotensin Converting Enzyme) inhibitors, which are useful in treatment of hypertension. Both compounds are members of a class of compounds disclosed and claimed in U.S. Pat. No. 4,344,949.

Quinapril and Moexipril will both form acid addition salts, such as hydrochloride. Also, since both quinapril and moexipril have a carboxylic acid moiety, both can be reacted with bases to form basic salts such as the sodium and magnesium salts.

As is the case with other similar compounds, quinapril and moexipril and their salts are susceptible to degradation by both cyclization and hydrolysis. Both types of degradation are accelerated by various excipients (i.e. inactive ingredients) used in the manufacture of pharmaceutical tablets. It has thus been found difficult to formulate tablets comprising salts of either quinapril or moexipril that are stable against such degradation.

U.S. Pat. No. 4,743,450 teaches that ACE inhibitors, and in particular quinapril and its acid addition salts, are stabilized in compositions that comprise both an alkaline compound and a saccharide. It is taught that the alkaline compound inhibits cyclization, and the saccharide inhibits hydrolysis. Magnesium, calcium and sodium compounds are said to be preferred as alkaline compound, and magnesium is most preferred.

Examples A and B in this patent both show tablets comprising quinapril hydrochloride as the active drug, magnesium carbonate as the alkaline compound, and lactose as the saccharide. Both examples also include gelatin as binder, crospovidone (also known as polyplasdone) as disintegrant, and magnesium stearate as lubricant.

There is also a publication by Gu et al, “Drug-Excipient Incompatibility Studies of the Dipeptide Angiotensin Converting Enzyme Inhibitor, Moexipril Hydrochloride: Dry Powder vs Wet Granulation”, Pharm Res. 7(4):370-383. This publication discloses that moexipril hydrochloride can be stabilized by making compositions comprising moexipril hydrochloride and an alkaline stabilizing agent selected from sodium bicarbonate, sodium carbonate and calcium carbonate. It is stated that the stabilization is accomplished only when the compositions are made by a wet granulation process. In the conclusion of the publication, it is postulated that the stabilization results from the neutralization of the acidic drug by the basic excipient at the outer surface of the granulated material. It is also stated that it is possible that a portion of the moexipril is converted to alkaline salts via granulation. It thus appears clear that Gu et al teaches that only a portion (if any) of the drug (and only that portion at the outer surface of the granules), may be converted to the alkaline salt, and that the stable product thus results entirely or primarily not from conversion to the alkaline salt, but from stabilization of the moexipril hydrochloride by the presence of the alkaline stabilizing compound in the final product.

Gu et al is thus consistent with the teaching of U.S. Pat. No. 4,743,450, which, as aforesaid, teaches stable compositions comprising the unstable drug, stabilized by the presence of an alkaline compound in the final composition.

Tablets containing a quinapril salt are sold in the United States and elsewhere under the tradename Accupril™ by Warner-Lambert Company. The labelling of these tablets indicates that the tablets contain quinapril hydrochloride, magnesium carbonate, lactose, gelatin, crospovidone and magnesium stearate.

Tablets containing a moexipril salt are sold in the United States and elsewhere under the tradename Univasc™ by Schwarz Pharma. The labelling of these tablets indicates that the tablets contain moexipril hydrochloride, magnesium oxide, lactose, gelatin, crospovidone and magnesium stearate.

The labelling thus indicates that both products are made in accordance with the teaching of U.S. Pat. No. 4,743,450 and Gu et al.

U.S. patent application Ser. Nos. 09/857,640 and 09/809,173 disclose that, in a tablet comprising a salt of quinapril or moexipril, the need to include an alkaline stabilizing compound in the tablet can be eliminated by using as the active drug the magnesium salt of quinapril or moexipril. The magnesium salt can be made by reacting the hydrochloride salt with a magnesium basic compound such as magnesium hydroxide, magnesium oxide, or magnesium carbonate in the presence of a solvent, and then evaporating the solvent. According to these patent applications, the levels of degradation products, and in particular the levels of the cyclization product, are substantially lower in tablets comprising the magnesium salt than in similar tablets comprising the hydrochloride salt, with no alkaline stabilizer.

In all of the examples in both of these patent applications, the tablets include lactose, which is a saccharide, as an excipient. ™-Registered trademark

It has subsequently been found that, even though the tablets of U.S. patent application Ser. Nos 09/857,640 and 09/809,173, which comprise the magnesium salt of quinapril or moexipril, are much more stable against cyclization than tablets comprising the hydrochloride salt, the rate of cyclization is reduced even further if the tablets also include an alkaline stabilizer. However, tablets containing a salt of quinapril or moexipril, even if the magnesium salt rather than the hydrochloride, might still be considered with the scope of the claims of U.S. Pat. No. 4,743,450 if they contain both an alkaline stabilizer and a saccharide.

In light of the foregoing, it is the objective of the present invention to enable tablets comprising salts of quinapril and moexipril which are as stable against both cyclization and hydrolysis as Accupril™ and Univasc™, but are not within the scope of the claims of U.S. Pat. No. 4,743,450, so as to provide an alternative for formulators.

It is also an objective to enable tablets with fewer ingredients than required by the teaching of U.S. Pat. No. 4,743,450.

As aforesaid, U.S. Pat. No. 4,743,450 teaches the use of both an alkaline compound to inhibit cyclization and a saccharide to inhibit hydrolysis. Since inclusion of an alkaline compound to inhibit cyclization is desirable, the objective of the present invention, more particularly, is to enable tablets that include an alkaline compound to inhibit cyclization, but do not include a saccharide to inhibit hydrolysis, and yet are still stabilized against hydrolysis.

### DESCRIPTION OF THE INVENTION

It has surprisingly been found that tablets which comprise a salt of quinapril or moexipril and also comprise an alkaline compound which inhibits cyclization can be further stabilized against hydrolysis without inclusion of a saccharide by including in the composition either an excipient that is not a saccharide but is water-soluble, or an excipient that is not a saccharide and is not water-soluble but absorbs water, or both.

The salt of quinapril or moexipril will preferably be the magnesium salt, i.e. magnesium di-quinapril or magnesium di-moexipril.

The alkaline stabilizer that is included in the tablets will preferably be a magnesium compound, and will most preferably be selected from magnesium hydroxide, magnesium oxide, and magnesium carbonate.

As aforesaid, the excipient that further stabilizes against hydrolysis will not be a saccharide, but will be either a compound that is water-soluble or a compound that is not water-soluble but absorbs water, or both.

Where the excipient selected is one that is water-soluble, it will preferably be a compound that also serves as a binder to increase tablet hardness, such as, for example, povidone or copolyvidone.

Where the excipient selected is one that is not water-soluble but absorbs water, it will preferably be one that also serves as a disintegrant to speed disintegration of the tablet after ingestion, such as, for example, crospovidone.

The amount of the said excipient or excipients that further stabilize against hydrolysis will preferably exceed five percent of the composition by weight, will more preferably exceed ten percent, and will even more preferably exceed twenty percent.

The tablets will preferably further comprise a lubricant such as, for example, magnesium stearate or zinc stearate.

The invention will be further understood from the following examples, which are intended to be illustrative but not limiting of the invention.

### EXAMPLES

### Example 1

A complex (i.e. mixture) of magnesium di-quinapril and magnesium chloride was made by reacting quinapril hydrochloride with magnesium hydroxide in a mixture of water and acetone (using an excess of magnesium hydroxide to ensure completeness of the reaction), filtering to remove the excess magnesium hydroxide, and then evaporating the water and acetone.

### Examples 2 to 5

Ingredients were then mixed together in the following proportions:

| Example No. | 2 | 3 | 4 | 5 | 6 | 7 |
|---|---|---|---|---|---|---|
| Complex of example 1 | 6.25 | 6.25 | 6.25 | 6.25 | 6.25 | 6.25 |
| Anhydrous lactose | 42.4 | 0 | 0 | 0 | 0 | 0 |
| Magnesium carbonate | 0 | 30 | 20 | 13.4 | 13.4 | 13.4 |
| Crospovidone | 1.0 | 13.4 | 23.4 | 30 | 10 | 0 |
| Povidone | 0 | 0 | 0 | 0 | 20 | 30 |
| Zinc stearate | 0.35 | 0.35 | 0.35 | 0.35 | 0.35 | 0.35 |
| | 50. | 50. | 50. | 50. | 50. | 50. |

For each of examples 2 to 7, the powder mixture was then compressed into tablets of weight 50 mg per tablet.

Since the mixture of example 2 comprises a saccharide (i.e. lactose) and does not comprise an alkaline compound, it is not an example of the present invention, but is included for comparison purposes.

Tablets of each of examples 2 to 7 were stored for two weeks at 40° C./75% R.H. (relative humidity) and then tested for degradation products.

The results were as follows:

| Example # | Cyclization Product | Hydrolysis Product |
|---|---|---|
| 2 | 5.04% | 12.45% |
| 3 | .55% | 10.58% |
| 4 | .54% | 9.22% |
| 5 | .42% | 8.62% |
| 6 | .37% | 8.41% |
| 7 | .27% | 8.32% |

From the above results, it can be seen that:

i) In examples 3 to 7 the amount of cyclization product is substantially less than in example 2, which confirms that the inclusion of an alkaline compound further reduces that rate of cyclization, even when the active drug is in the form of the magnesium salt, and

ii) In examples 3 to 7 the amount of hydrolysis product is less than in example 2, even though example 2 contained a saccharide and examples 3 to 7 did not. This confirms that crospovidone and povidone are as effective as lactose in reducing the rate of hydrolysis.

### Examples 8 to 12

Ingredients were used in the proportions as follows:

| Example No. | 8 | 9 | 10 | 11 | 12 |
|---|---|---|---|---|---|
| Moexipril hydrochloride | 7.5 | 7.5 | 7.5 | 7.5 | 7.5 |
| Magnesium carbonate | 71.5 | 50.0 | 21.5 | 21.0 | 0 |
| Crospovidone | 0 | 21.5 | 50.0 | 42.0 | 71.5 |
| Povidone | 0 | 0 | 0 | 8.0 | 0 |
| Red Ferric Oxide | 0 | 0 | 0 | 0.5 | 0 |
| Water | 71. | 80. | 89. | 90. | 84. |
| Zinc stearate | 1.0 | 1.0 | 1.0 | 1.0 | 1.0 |
| Total excluding water | 80. | 80. | 80. | 80. | 80. |

For each example, the ingredients other than water and zinc stearate were mixed together, and then the water was added to make a damp mass. The damp mass was then further well mixed, following which it was dried for several hours in an oven at 40° C. The dried mass was then milled into fine granules, the zinc stearate was added, and the mixture was compressed into tablets of weight 80 mg each.

In each of examples 8 to 11, during the process the mass was wet enough for long enough to allow a complete or almost complete reaction of the moexipril hydrochloride with magnesium carbonate to convert the moexipril hydrochloride to magnesium di-moexipril. In example 12 there was no magnesium carbonate with which the moexipril hydrochloride could react, so the final tablets contained the drug as moexipril hydrochloride.

As example 8 contains no excipient to stabilize against hydrolysis and example 12 contains no alkaline compound to stabilize against cyclization, neither is an example of the present invention, and both are included for comparison purposes.

Tablets of each of examples 8 to 12 were stored for two weeks at 40° C./75% R.H. and then tested for degradation products.

The results were as follows:

| Example # | Cyclization Product | Hydrolysis Product |
|---|---|---|
| 8 | 0.08% | 6.72% |
| 9 | 0.07% | 3.01% |
| 10 | 0.06% | 2.88% |
| 11 | 0.07% | 3.68% |
| 12 | 62.6% | 0.19% |

From the above results it can be seen that:

i) Example 12 was very unstable against cyclization because no alkaline compound was included; and

ii) In examples 9, 10 and 11, the amount of hydrolysis product is less than in example 8, because of the inclusion of crospovidone and/or povidone in examples 9, 10 and 11.

1. A tablet which comprises a salt of quinapril or moexipril and an alkaline compound to inhibit cyclization, and which does not comprise a saccharide, and which further comprises one or more excipients that improve stability against hydrolysis, selected from compounds that are water-soluble or are not water-soluble but absorb water.

2. A tablet of claim 1 that comprises a salt of quinapril.

3. A tablet of claim 1 that comprises a salt of moexipril.

4. A tablet of any of claims 1 to 3, wherein the salt is the magnesium salt.

5. A tablet of any of claims 1 to 4, wherein the alkaline compound is a magnesium compound.

6. A tablet of any of claim 5, wherein the alkaline compound is selected from the group consisting of magnesium hydroxide, magnesium oxide and magnesium carbonate.

7. A tablet of any of claims 1 to 6, wherein the said one or more excipients comprise a compound that is water-soluble.

8. A tablet of claim 7 wherein the said compound also serves as a binder to increase tablet hardness.

9. A tablet of claim 7 or 8, wherein the said compound is povidone.

10. A tablet of claim 7 or 8, wherein the said compound is copolyvidone.

11. A tablet of any of claims 1 to 6, wherein the said one or more excipients comprise a compound that is not water-soluble but absorbs water.

12. A tablet of claim 11, wherein the said compound also serves as a disintegrant.

13. A tablet of claim 12, wherein the said compound is crospovidone.

14. A tablet of any of claims 1 to 6 wherein said excipient or excipients comprise both a compound that is water-soluble and a compound that is not water-soluble but absorbs water.

15. A tablet of claim 14 that comprises crospovidone.

16. A tablet of claim 14 that comprises povidone or copolyvidone.

17. A tablet of claim 14 that comprises both crospovidone and povidone.

18. A tablet of claim 14 that comprises both crospovidone and copolyvidone.

19. A tablet of any of claims 1 to 18, wherein the amount of the said one or more excipients exceeds five percent of the tablet by weight.

20. A tablet of claim 14, wherein the amount of the said one or more excipients exceeds ten percent of the tablet by weight.

21. A tablet of claim 15, wherein the amount of the said one or more excipients exceeds twenty percent of the tablet by weight.
